(19) United States
(12) Patent Application Publication
(10) Pub. No.: US 2009/0209656 A1
(43) Pub. Date: Aug. 20, 2009

(54) DISINFECTION/SANITATION METHOD WHERE THE MATERIAL IS TREATED WITH A SOLUTION COMPRISING BENZYL ALCOHOL AND C2-3 ALCOHOL

(75) Inventor: Hamid Houshmand, Uppsala (SE)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE BIO-SCIENCES AB, Uppsala (SE)

(21) Appl. No.: 12/300,641

(22) PCT Filed: Jun. 12, 2007
(86) PCT No.: PCT/SE2007/000575

§ 371 Date: Nov. 13, 2008

(30) Foreign Application Priority Data
Jul. 6, 2006 (SE) 0601521-8

(51) Int. Cl. A61K 31/045

(52) U.S. Cl. 514/730

(57) ABSTRACT

The present invention relates to a method for disinfection or sanitation of different material, especially sensitive material, such as chromatographic media with various sensitive ligands. The invention also relates to a composition for use in such a method. The method for disinfection/sanitation, comprises exposing the material to be treated to a solution comprising 5-10% benzyl alcohol and 10-30% C₂₋₃ alcohol.

### CROSS-REFERENCE TO RELATED APPLICATIONS

This application is a filing under 35 U.S.C. § 371 and claims priority to international patent application number PCT/SE2007/000575 filed Jun. 12, 2007, published on Jan. 10, 2008, as WO 2008/004931, which claims priority to patent application number 0601521-8 filed in Sweden on Jul. 6, 2006.

### FIELD OF THE INVENTION

The present invention relates to a method for disinfection or sanitation of different material, especially sensitive material, particularly chromatographic media with various sensitive ligands, such as proteinaceous ligands. The invention also relates to a composition for use in such a method.

### BACKGROUND OF THE INVENTION

Contamination with bacteria and other microbes is an often encountered problem within many biotechnological and biomedical applications.

Methods for killing bacterial cells and other microbes involve application of heat, addition of solvents or of strong acids and bases, or the use of electricity or irradiation. However, almost all sterilization methods such as gamma irradiation and autoclaving and most effective disinfectants and sanitation reagents such as strong acids or alkalis, quaternary ammonium compounds, halogen-containing compounds, oxidizing agents, and phenols and related compounds are harmful and/or toxic.

Disinfection of chromatographic media is very cumbersome, particularly disinfection of proteinaceous media, i.e. chromatographic media provided with proteinaceous ligands, such as various affinity chromatography media. The protein ligand on the chromatographic media is extremely sensitive to harsh conditions. These kind of media do not tolerate any known effective disinfectant reagent or strong sanitation reagents.

U.S. Pat. No. 4,966,844 describes a method for microbial cell killing. This method describes use of 0.5-10% benzyl alcohol for killing of the host cells in processes for recovery of recombinant proteins from fermentation cultures. This method avoids degradation of pH- and temperature sensitive products.

Hitherto there exists no method for efficient disinfection/sanitation of sensitive material, such as chromatographic media.

### SUMMARY OF THE INVENTION

The present invention provides a new disinfection/sanitation or conservation method and a composition for use in this method.

In a first aspect, the invention relates to a method for disinfection or sanitation, comprising exposing the material to be disinfected to a solution comprising 5-10% benzyl alcohol and 10-30% C₂₋₃ alcohol.

Preferably, the material to be sterilised is a chromatography media, chromatography column or filter.

The C₂₋₃ alcohol may be isopropanol, propanol or ethanol, preferably isopropanol.

Preferably, the sterilisation is performed at pH 4-6 and in room temperature (18-24° C.).

In a second aspect, the invention relates to a composition for disinfection, comprising a solution comprising 5-10% benzyl alcohol and 10-30% C₂₋₃ alcohol.

A preferred composition for disinfection comprises 5-10% benzyl alcohol and 10-30% isopropanol.

In a third aspect, the invention relates to use of a composition as above for disinfection/sanitation or conservation of a chromatography media, column or filter, preferably a chromatographic media comprising proteinaceous ligands.

### DETAILED DESCRIPTION OF THE INVENTION

The present invention provides a new, safe, and highly efficient and harmless method for disinfection/sanitation of chromatography columns/media and filters. The method is based on an exposure of the chromatography media/column/filter to a solution of 5-10% benzyl alcohol, 10-30% isopropanol (alternatively ethanol or propanol), pH 4-6 at room temperature (18-24° C.).

Results from antimicrobial challenging tests indicate that this reagent according to the invention is capable of an efficient disinfection/sanitation. The method/reagent has also been shown to be very gentle and harmless to susceptible media, such as MABSELECT™ and MABSELECT SURE™.

Benzyl alcohol exhibits a very low acute toxicity. The LD50 of benzyl alcohol is 1610 mg/kg body weight. The long-term toxicity study of benzyl alcohol indicates a NOAEL >400 mg/kg body weight/day implying a non-significant toxic effect in long-term exposure. Benzyl alcohol has a high flash point (213° F.). This makes usage of benzyl alcohol less of a fire hazard in industrial settings.

The composition of the invention has a mixture of ingredients and properties which makes it suitable for disinfection/sanitation as well as conservation of sensitive products, such as chromatographic media.

The disinfection/sanitation method according to the invention is widely applicable and may be used for chromatography medias intended for any purpose, such as cell separation and food separation.

The disinfection method is suite also for disposable columns and filters. The disposable columns/filters give the advantage of being ready-to-use without any need of cleaning and sanitation. This implies columns/filters with good sterility or at least free of microbes.

Thus, a method for a highly efficient and harmless disinfection/sanitation of chromatography columns/media/filters is provided by the present invention.

### EXAMPLES

The present invention will be described in more detail by way of examples, which however are in no way intended to limit the scope of the present invention as defined by the appended claims. All references given below or elsewhere in the present specification are hereby included herein by reference.

A composition according to the invention comprising a solution comprising 10% benzyl alcohol and 30% isopropanol was used to sanitize contaminated MABSELECT™ media (GE Healthcare AB, Uppsala Sweden).

To the media prepared in 50% slurry with 10% benzyl alcohol, 30% isopropanol solution was added comprising 10⁶/ml of the following microorganisms: Staphylococcus aureus (ATCC 6538P) or Aspergillus niger (ATCC 16404).

The media was incubated with the composition of the invention for 1 hour in RT. The pH of the composition was 5.

Following the incubation, the media was free from substantially all added microorganisms. The results obtained showed a very efficient reduction of Staphylococcus aureus and Aspergillus niger from 10⁶/ml to the detection limit (0 CFU /ml) within one hour.

The results obtained from challenging tests implies that the following microorganisms: E.coli, Pseudomas aeruginosa, Candida albicans would also be killed by the composition according to the invention at least with same efficiency as Staphylococcus aureus and Aspergillus niger.

The above examples illustrate specific aspects of the present invention and are not intended to limit the scope thereof in any respect and should not be so construed. Those skilled in the art having the benefit of the teachings of the present invention as set forth above, can effect numerous modifications thereto. These modifications are to be construed as being encompassed within the scope of the present invention as set forth in the appended claims.

1. A method for disinfection/sanitation, comprising exposing the material to be treated to a solution comprising 5-10% benzyl alcohol and 10-30% C₂₋₃ alcohol.

2. The method of claim 1, wherein the material to be sanitized is a chromatography media, chromatography column or filter.

3. The method of claim 1, wherein the C₂₋₃ alcohol is isopropanol, propanol or ethanol.

4. The method of claim 1, wherein the disinfection is performed at pH 4-6.

5. The method of claim 1, wherein the disinfection is performed at room temperature (18-24° C.).

6. A composition for disinfection/sanitation, comprising a solution comprising 5-10% benzyl alcohol and 10-30% C₂₋₃ alcohol.

7. A composition for disinfection/sanitation, comprising a solution comprising 5-10% benzyl alcohol and 10-30% isopropanol.

8. The composition of claim 7, comprising a solution comprising 10% benzyl alcohol and 30% isopropanol.

9-10. (canceled)
